(19) United States
(12) Patent Application Publication
(10) Pub. No.: US 2025/0366891 A1
(43) Pub. Date: Dec. 4, 2025

(54) FRICTION-FIT MODULAR POLYAXIAL SCREW ASSEMBLIES

(71) Applicant: Orthofix US LLC, Lewisville, TX (US)

(72) Inventor: Jonathon James Gerding, Lewisville, TX (US)

(21) Appl. No.: 19/191,875

(22) Filed: Apr. 28, 2025

Related U.S. Application Data
(60) Provisional application No. 63/652,393, filed on May 28, 2024.

(51) Int. Cl. A61B 17/70; A61B 17/00

(52) U.S. Cl. CPC A61B 17/7032; A61B 17/7035; A61B 17/7043; A61B 2017/00477; A61B 2017/00862

(57) ABSTRACT

Implantable devices and assemblies are described that provide a friction fit between a receiver and a fastener, such as a bone screw. The friction is created between a screw head, a retention ring, and a pressure insert, stabilizing the receiver's orientation before locking with a set screw. This mechanism facilitates manual alignment of the receiver in a way that retains the orientation of the receiver after it has been moved and oriented at a desired setting. The devices and assemblies allow for modular assembly before or during spinal fixation procedures, accommodating various screw characteristics. The frictional force may be facilitated by resilient tabs on the pressure insert, which engage a recess and/or a ridge in the receiver. This engagement translates into a downward spring force of the pressure insert on the head of the bone screw, which helps provide the friction between the head of the bone screw and the receiver assembly.

### CROSS-REFERENCE TO RELATED APPLICATIONS

This patent application claims the benefit of the filing date of U.S. Provisional Patent Application 63/652,393, filed May 28, 2024, and titled, Friction-Fit Modular Polyaxial Screw Assemblies, which is incorporated herein by reference, in its entirety.

### TECHNICAL FIELD

This disclosure is generally directed to friction-fit devices and assemblies attachable to bone fasteners for implantation in an anatomy of a patient. For instance, one or more implantable assemblies including a receiver body coupled to a bone screw may be coupled to a connecting rod to retain one or more vertebrae in a desired relationship.

### BACKGROUND

Various systems for connecting fasteners (e.g., pedicle screws) to elongated supports (e.g., fixation rods) for the purposes of vertebral fixation have been proposed. Although described with reference to vertebral or spinal fixation, it should be appreciated that the systems described herein may be similarly applicable to other bone structures as well.

Generally, fixation systems include a receiver (or “receiver body” or “head”) which is attachable to both a fastener and a fixation rod to retain the rod in fixed relation to the fastener, and in turn, a vertebra into which the fastener is secured. Traditional receiver assemblies include a receiver and a fastener for attachment of fixation rod to a vertebra. A physician may use multiple receiver assemblies and/or multiple rods to secure the vertebrae in a desired spatial relationship. In some installations, a first rod may extend along a first side of a patient's spine and engage a first plurality of fastener assemblies each implanted in a different vertebra, and a second rod may extend along a second side of the patient's spine and engage a second plurality of fastener assemblies.

In some instances, a receiver assembly may come preassembled such that the receiver and fastener are preselected and attached to one another by the manufacturer. The assembly of the fastener and the receiver may involve special tools and trained technicians such that assembly by the physician, nurse, or surgical technician is impractical. Accordingly, the surgeon or technician may select a receiver and fastener assembly from a plurality of receiver and fastener assemblies based on the patient's anatomy and/or indications. Thus, the surgeon may be limited based on the variety of selections available at the time of surgery.

During a spinal fixation surgery, the receiver and fastener assemblies may be inserted through the patient's tissue via a surgical opening or ingress. The fasteners of each assembly may be driven into the patient's vertebra at desired locations. A connecting rod is then positioned through each receiver and the receivers and connecting rod are fixed in place by set screws or compression screws in each receiver. In order to position the connecting rod through each receiver, the receivers are oriented in alignment so that the connecting rod can be inserted through a channel or slot of each receiver. The alignment of the receivers can be a complicated part of the procedure. For example, gravity may cause the receivers to droop or slip out of alignment. Accordingly, the procedure may involve repositioning and/or reorienting one or more receivers multiple times before the connecting rod is successfully positioned through each receiver.

### SUMMARY

The present disclosure describes implantable devices and assemblies that provide a friction fit between a receiver and a fastener (e.g., bone screw). For example, a frictional force may be applied between a screw head and a retention ring, and another frictional force may be applied between the screw head and a pressure insert. The frictional force and contact maintains and stabilizes an orientation of a receiver relative to the screw head before the position is fixed by a set screw. This friction fit mechanism reduces or prevents drooping or slipping of the receiver out of alignment when the fastener is implanted into the bone and prior to locking with a set screw. Further, the implantable devices of the present disclosure may allow for modular assembly before or during a spinal fixation procedure. For example, the implantable device may allow for bottom-side loading of the screw head up into the receiver so that various screws having various characteristics (e.g., length, diameter, etc.) can be coupled to the receiver body before or after the bone screw has been implanted into bone. The frictional force may be facilitated by one or more resilient tabs of the pressure insert which are configured to flex or deflect in response to longitudinal movement of the pressure insert relative to the receiver. The resilient tabs may engage a ramped or sloped surface of a tapered recess in the receiver body, such that longitudinal movement of the pressure insert is translated into compressing or flexing the resilient tabs inward. The ramped or sloped surface may be described as a camming surface, as longitudinal travel of the pressure insert facilitates flexing of the tabs inward in a direction transverse to the pressure insert's longitudinal axis.

According to one embodiment of the present disclosure, a fastener assembly for a spinal fixation system, comprises: a bone shank comprising a head portion and a distal threaded portion configured to be implanted into bone; a receiver comprising: an axial bore extending longitudinally through the receiver from a proximal opening of the receiver to a distal opening; and an interior surface surrounding a portion of the axial bore, the interior surface defining a tapered recess, the tapered recess increasing in depth toward a distal end of the tapered recess; a pressure insert disposed at least partly within the axial bore, the pressure insert comprising: a saddle configured to seat a fixation rod; a distally-facing concave surface configured to contact the head portion of the bone shank; and a resilient tab projecting proximally and outward from an exterior cylindrical surface of the pressure insert; wherein the pressure insert is longitudinally displaceable in the axial bore from a first longitudinal position to a second longitudinal position, wherein longitudinal displacement of the pressure insert to the second longitudinal position is resisted by a spring force facilitated by rounded face of the resilient tab engaging the tapered recess, and wherein, when the pressure insert returns to the first longitudinal position, the spring force urges the pressure insert distally against the head portion of the bone shank to facilitate a friction-fit engagement between the head portion of the bone shank and the receiver.

In some embodiments, the receiver further comprises a chamber disposed adjacent to the distal opening and configured to receive the head portion of the bone shank therein; the fastener assembly further comprises a split retainer ring disposed in the chamber; and the friction-fit engagement is at least partially between the head portion of the bone shank and the split retainer ring.

In some embodiments, the head portion of the bone shank comprises a spherical surface, and the split retainer ring comprises an interior concave surface configured to contact the spherical surface to thereby provide a pivotable relationship between the head portion of the bone shank and the interior concave surface of the split retainer ring. In some embodiments, the receiver comprises an interior conical surface defining the chamber, and the split retainer ring further comprises an outer conical surface. In some embodiments, when the pressure insert is in the second longitudinal position, the outer conical surface abuts the interior conical surface of the receiver.

In some embodiments, the fastener assembly further includes a compression screw configured to be threadably received into an upper opening of the receiver along a longitudinal axis of the receiver, and wherein the compression screw is configured to compress the fixation rod against the pressure insert, which in turn locks the fastener assembly by simultaneously urging: the spherical surface of the head portion of the bone shank against the interior concave surface of the split retainer ring; and the outer conical surface of the split retainer ring against the interior conical surface of the receiver.

In some embodiments, the resilient tab comprises a rounded face contacting the tapered recess. In some embodiments, the rounded face is configured to make point contact with the tapered recess. In some embodiments, the fastener assembly further includes a second resilient tab disposed in the tapered recess, wherein the tapered recess comprises a conical bore. In some embodiments, the tapered recess comprises a planar ramped surface configured to contact the resilient tab, and the interior surface surrounding the axial bore further defines a second tapered recess comprising a second planar ramped surface. In some embodiments, the pressure insert further comprises a second resilient tab disposed in the second tapered recess and configured to make sliding contact with the second planar ramped surface.

According to one embodiment of the present disclosure, a receiver for a polyaxial bone screw assembly is described, the receiver comprising: a body, comprising: an axial bore extending longitudinally through the receiver from a proximal opening of the receiver to a distal opening; a chamber disposed adjacent to the distal opening and configured to receive a head portion of a bone shank therein; and an interior surface surrounding a portion of the axial bore, the interior surface defining a tapered recess, the tapered recess increasing in depth toward a distal end of the tapered recess; a split retainer ring disposed in the chamber; and a pressure insert disposed at least partly within the axial bore, the pressure insert comprising: a first surface configured to seat a fixation rod; a distally-facing concave surface configured to contact the head portion of the bone shank; and a resilient tab projecting proximally and outward from an exterior cylindrical surface of the pressure insert, the resilient tab comprising a rounded face engaging the tapered recess, wherein the pressure insert is longitudinally displaceable in the axial bore from a first longitudinal position to a second longitudinal position, wherein longitudinal displacement of the pressure insert to the second longitudinal position causes the resilient tab engaging the tapered recess to deflect, thereby providing a spring resistance in a distal direction.

In some embodiments, the split retainer ring comprises an interior concave surface. In some embodiments, the body further comprises a conical bore, wherein: the split retainer ring further comprises an outer conical surface, and when the pressure insert is in the second longitudinal position, the outer conical surface abuts the conical bore. In some embodiments, the resilient tab comprises a rounded face contacting the tapered recess. In some embodiments, the rounded face is configured to make point contact with the tapered recess. In some embodiments, a second resilient tab is disposed in the tapered recess, wherein the tapered recess comprises a conical bore. In some embodiments, the tapered recess comprises a planar ramped surface configured to contact the resilient tab; the interior surface surrounding the axial bore further defines a second tapered recess comprising a second planar ramped surface. In some embodiments, the pressure insert further comprises a second resilient tab disposed in the second tapered recess and configured to make sliding contact with the second planar ramped surface.

According to one embodiment of the present disclosure, a receiver body for a polyaxial bone screw assembly is described, the receiver body comprising: an axial bore extending longitudinally through the receiver body from a proximal opening of the receiver body to a distal opening; a first tapered chamber disposed adjacent to the distal opening and configured to receive a head portion of a bone shank therein, wherein the first tapered chamber increases in width toward a top portion of the first tapered chamber; a second tapered chamber disposed proximal to the first tapered chamber, wherein the second tapered chamber increases in width toward a bottom portion of the second tapered chamber; and a channel defined in an upper portion of the receiver body and configured to receive a fixation rod therein.

In some embodiments, the first tapered chamber comprises a first conical bore. In some embodiments, the second tapered chamber comprises a second conical bore. In some embodiments, the second tapered chamber comprises a first planar ramped surface, and the receiver body comprises a third tapered chamber comprising a second planar ramped surface.

According to one embodiment of the present disclosure, a method for assembling a polyaxial fastener assembly is described, comprising: inserting a head portion of a bone shank through a distal opening of a receiver assembly and into a chamber of the receiver assembly, wherein the receiver assembly comprises: a receiver body, comprising: the distal opening; a conical interior surface disposed about the chamber; an axial bore extending longitudinally through the receiver body; and a tapered recess defined in a surface of the axial bore; a split retainer ring disposed in the chamber; and a pressure insert disposed at least partially within the axial bore, the pressure insert comprising: a resilient tab projecting proximally and outward from an exterior surface of the pressure insert, the resilient tab comprising a rounded face engaging the tapered recess, wherein the inserting comprises pushing the head portion through the distal opening of the receiver body and through the split retainer ring to: cause the split retainer ring to elastically expand about the head portion, and thereafter to collapse about a neck of the bone shank to retain the head portion within the chamber in a pivotable relationship with the receiver body; and cause the pressure insert to move to a first longitudinal position; wherein the method further comprises: releasing a distally-directed force on the receiver assembly to allow the pressure insert to return to a second longitudinal position, wherein, in the second longitudinal position, a spring force provided by the resilient tab urges the pressure insert distally against the head portion of the bone shank to facilitate a friction-fit engagement between the head portion of the bone shank and the split retainer ring.

According to one embodiment of the present disclosure, a method for assembling a polyaxial fastener assembly is described, comprising: providing a receiver body, comprising: an axial bore extending longitudinally through the receiver body from a proximal opening of the receiver body to a distal opening, the distal opening comprising a width; a first tapered chamber disposed adjacent to the distal opening, wherein the first tapered chamber increases in diameter toward a top portion of the first tapered chamber; and a second tapered chamber disposed proximal to the first tapered chamber, wherein the second tapered chamber increases in diameter toward a bottom portion of the second tapered chamber; inserting a pressure insert into the receiver body through one of the proximal opening or the distal opening, wherein the inserting the pressure insert comprises engaging at least one resilient tab of the pressure insert into the second tapered chamber; and inserting a split retainer ring through the distal opening such that a conical outer surface of the split retainer ring rests against a conical surface of the second tapered chamber, wherein the inserting the split retainer ring comprises compressing the split retainer ring from a first width to a second width, wherein the second width of the split retainer ring is smaller than the width of the distal opening.

According to another embodiment of the present disclosure, a fastener kit for a spinal fixation system comprises: a bone shank comprising a head portion and a distal threaded portion configured to be implanted into bone; a receiver comprising: an axial bore extending longitudinally through the receiver from a proximal opening of the receiver to a distal opening; an interior surface surrounding a portion of the axial bore, the interior surface defining a tapered recess, the tapered recess increasing in depth toward a distal end of the tapered recess; and a lower tapered chamber adjacent the distal opening; a pressure insert configured to be positioned in the axial bore, the pressure insert comprising: a saddle configured to seat a fixation rod; a distally-facing concave surface; and a resilient tab projecting proximally and outward from an exterior cylindrical surface of the pressure insert, wherein the resilient tab is sized and shaped to extend at least partially within the tapered recess of the receiver; a split retainer ring comprising an outer tapered surface and configured to be positioned within the lower tapered chamber.

These and other objects, features and advantages of this invention will become apparent from the following detailed description of the various aspects and principles of the invention taken in conjunction with the accompanying drawings.

### BRIEF DESCRIPTION OF THE DRAWINGS

The accompanying drawings, which are incorporated in and constitute a part of the specification, illustrate embodiments of the invention and, together with the detailed description herein, serve to explain the principles of the invention. The drawings are only for purposes of illustrating examples and are not to be construed as limiting the invention. It is emphasized that, in accordance with the standard practice in the industry, various features are not drawn to scale. In fact, the dimensions of the various features may be arbitrarily increased or reduced for clarity of discussion.

FIG. 1 is a perspective view of a spinal fixation system including friction-fit pedicle screw assemblies and a connecting rod in accordance with an embodiment of the present disclosure.

FIG. 2A is a perspective view of a receiver for a friction-fit pedicle screw assembly in accordance with an embodiment of the present disclosure.

FIG. 2B is an exploded view of the receiver in FIG. 2A in accordance with an embodiment of the present disclosure.

FIG. 3A is a side perspective view of the receiver in FIG. 2A with a transparent body.

FIG. 3B is a bottom perspective view of the receiver in FIG. 2A with a transparent body.

FIG. 4A is a cross-sectional view of a receiver for a friction-fit pedicle screw assembly in accordance with an embodiment of the present disclosure.

FIG. 4B is a cross-sectional view of a pedicle screw assembly, including the receiver shown in FIG. 4A and a screw, in accordance with an embodiment of the present disclosure.

FIG. 4C is a cross-sectional view of a pedicle screw assembly, including the receiver shown in FIG. 4A and a screw, in accordance with an embodiment of the present disclosure.

FIG. 4D is a cross-sectional view of a pedicle screw assembly, including the

receiver shown in FIG. 4A and a screw, in accordance with an embodiment of the present disclosure.

FIG. 5A is a front view of a screw for use in a friction-fit pedicle screw assembly in accordance with an embodiment of the present disclosure.

FIG. 5B is a front view of a screw for use in a friction-fit pedicle screw assembly in accordance with an embodiment of the present disclosure.

FIG. 5C is a front view of a screw for use in a friction-fit pedicle screw assembly in accordance with an embodiment of the present disclosure.

FIG. 6 is a flow chart illustrating a method of implanting a friction-fit pedicle screw assembly in accordance with an embodiment of the present disclosure.

FIG. 7 is a flow chart illustrating a method of assembling a polyaxial friction fit screw assembly in accordance with an embodiment of the present disclosure.

FIG. 8 is a flow chart illustrating a method of assembling a polyaxial friction fit screw assembly in accordance with an embodiment of the present disclosure.

FIG. 9A is a cross-sectional view of a receiver for a friction-fit pedicle screw assembly in accordance with an embodiment of the present disclosure.

FIG. 9B is a cross-sectional perspective view of a receiver for a friction-fit pedicle screw assembly in accordance with an embodiment of the present disclosure.

FIG. 9C is a cross-sectional view of a receiver for a friction-fit pedicle screw assembly shown at three stages of assembly in accordance with an embodiment of the present disclosure.

### DETAILED DESCRIPTION

For the purpose of promoting an understanding of the principles of the present disclosure, reference will now be made to the implementations illustrated in the drawings and specific language will be used to describe them. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In addition, this disclosure describes some elements or features in detail with respect to one or more implementations or figures, when those same elements or features appear in subsequent figures, without such a high level of detail. It is fully contemplated that the features, components, and/or steps described with respect to one or more implementations or figures may be combined with the features, components, and/or steps described with respect to other implementations or figures of the present disclosure. For simplicity, in some instances the same or similar reference numbers are used throughout the drawings to refer to the same or like parts.

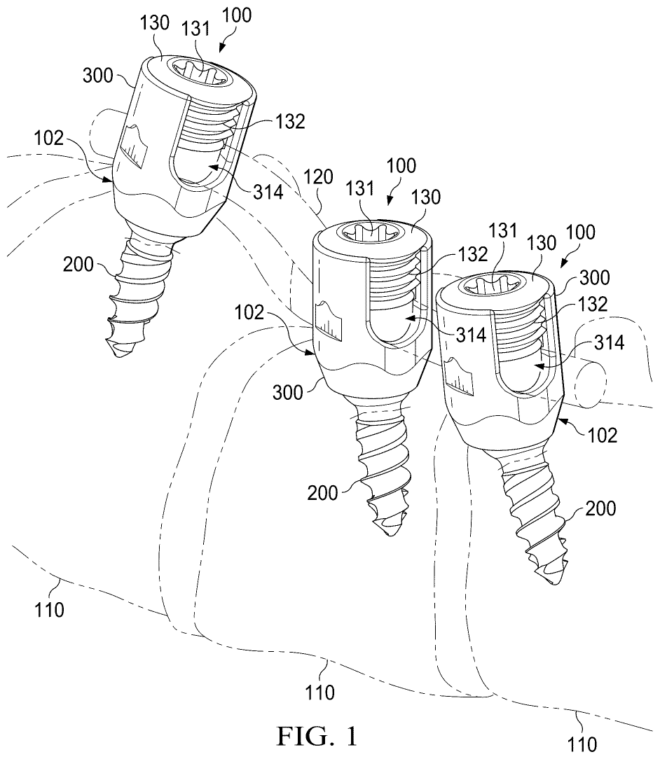

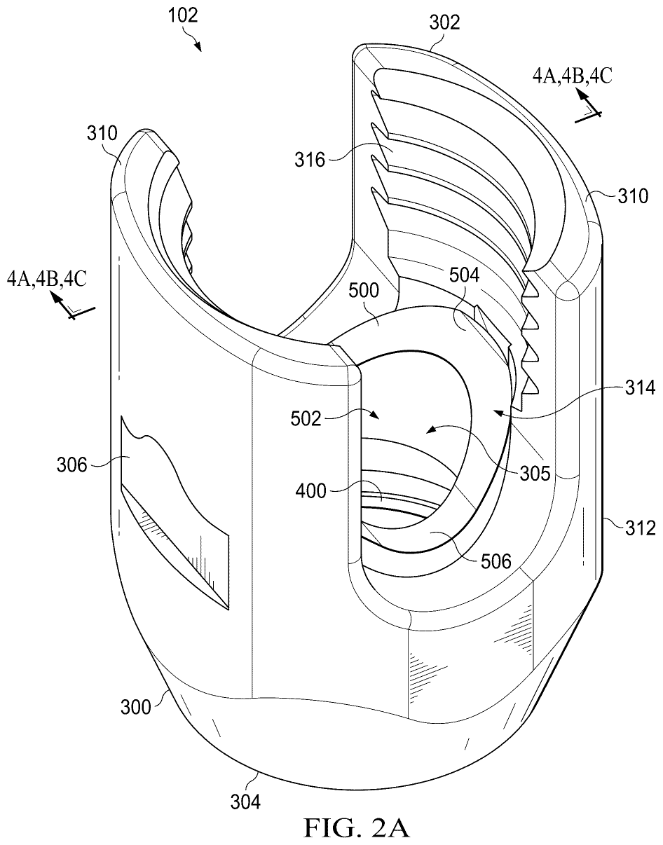

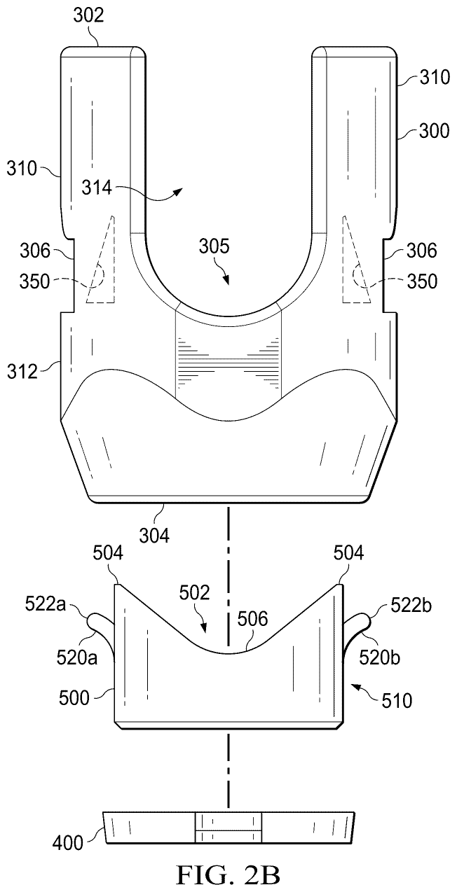

FIG. 1 is a perspective view of a plurality of pedicle screw assemblies 100 including a plurality of implantable receivers 102 coupled to respective vertebrae 110 of a patient's spine by a plurality of screws 200. Each implantable receiver 102 in FIG. 1 includes a body 300, and may further include a retainer ring (400, FIG. 2B), and a pressure cap or pressure insert (500, FIG. 2B) as described in more detail below. The receivers 102 are coupled to one another by a rod 120 positioned in U-shaped slots 314 or saddles of the receivers 102. The rod 120 may be sized, shaped (e.g., bent, curved), and otherwise structurally configured to correct a spinal deformity, and/or to retain the vertebrae 110 in a fixed position. The positions and orientations of the receivers 102 relative to the rod 120 and the bone screws 200 may be fixed or otherwise retained by the set screws 130. For example, the bone screws 200 may be coupled to the receivers 102 in a multi-axial relationship such that the bone screws 200 may be rotated about at least one axis relative to the respective receiver 102. For example, in some aspects, one or more of the bone screws 200 may include spherical, semi-spherical, or otherwise round screw heads (not shown) seated within the receiver 102. The receivers 102 may be configured to rotate, tilt, swivel, twist, and/or otherwise move relative to the screw heads of the bone screws 200. With the bone screws 200 fixed to the vertebrae 110, a physician may move the receivers 102 into the orientation shown in FIG. 1 to receive the rod 120. The orientations of the receivers 102 relative to one another may be maintained by the friction-fit coupling to the bone screws 200 as the physician guides the rod 120 through the slots 314. With the rod 120 in the slots 314, and with the desired overhang of the rod 120 from the outermost receivers 102, the set screws 130 can be tightened down to compress rod 120 and the screw heads of the screws 200 against the base of the receivers 102 to fix the position and orientation of the receivers 102 relative to the rod 120 and bone screws 200. The set screws 130 may be any appropriate shape. For example, the set screws 130 may include a drive feature 131 that receives an instrument for tightening the set screw 130 to compress the rod 120 and the screw heads of the screws 200. The drive feature 131 may be hexalobe shaped as shown in the illustrated embodiment, or may be any other appropriate shape including hexagonal, square, or triangular. Additionally, the set screws 130 may have threading 132 along part or all of the length. The threading 132 of the set screw 130 may threadably engage the receivers 102.

FIG. 2A is a perspective view of a receiver 102 according to an embodiment of the present disclosure. FIG. 2B shows an exploded view of the receiver 102 shown in FIG. 2A. Referring generally to FIGS. 2A and 2B, the receiver 102 may be similar or identical to the receivers 102 shown in FIG. 1, in some aspects. The receiver 102 includes a body 300, a retainer ring 400 or lock ring, and a pressure cap 500. The body 300 has a top end 302 for receiving a set screw (e.g., 130, FIG. 1) and a rod (e.g., 120, FIG. 1) and a bottom end 304 for receiving a screw head. The body 300 may further comprise an opening or axial bore 305. The opening (or axial bore) 305 may pass from the top end 302 to the bottom end 304. In the illustrated embodiment, the body 300 is tulip-shaped, meaning the body 300 has two arms 310 on either side of the body 300 that extend from a base 312 of the body 300 to the top 302. The arms 310 define a channel or U-shaped slot 314 for seating a rod. The arms 310 may be referred to as sidewalls, wings, or any other suitable term. The body 300 is configured to receive a connecting rod via the U-shaped slot 314. Moreover, the body 300 further includes internal threads 316 on the interior surfaces of the arms 310. The threads 316 may be configured to engage corresponding threads on a set screw (e.g., 130, FIG. 1). The set screw may be tightened down into the body 300 to compress the connecting rod onto the pressure cap 500. Compressing the pressure cap 500 may also cause the pressure cap 500 to apply additional pressure onto the screw head 210 of the screw 200 to fix the receiver 102 in a desired position and orientation.

The body 300 also has two engagement features 306 that may provide for releasable engagement with a tool for inserting, positioning, and/or removing the receiver 102. For example, the engagement features 306 may provide for releasable engagement with a tool for inserting the subassembly including the receiver 102 and the connected screw 200, and driving the screw 200 into the patient's bone (e.g., vertebra). In the illustrated embodiment, the engagement feature 306 is centered with the arm 310. It will be understood that the other arm 310 may also include an engagement feature similar or identical to the engagement feature 306. The engagement feature 306 on the other arm 310 may also be centered on the arm 310. The centering of the engagement feature 306 may be beneficial for robust engagement with the insertion tool. For example, the centered placement of the engagement feature 306 may allow for a deeper groove or impression of the engagement feature 306 into the arm 310. In another aspect, the top end 302 of the body 300 may be associated with a frangible portion or breaking line of the body 300. For example, in some embodiments, the body 300 may be integrally formed with extension portions or tower portions extending proximally from the top end 302. The area of the body 300 comprising the top end 302 may comprise a weakened portion.

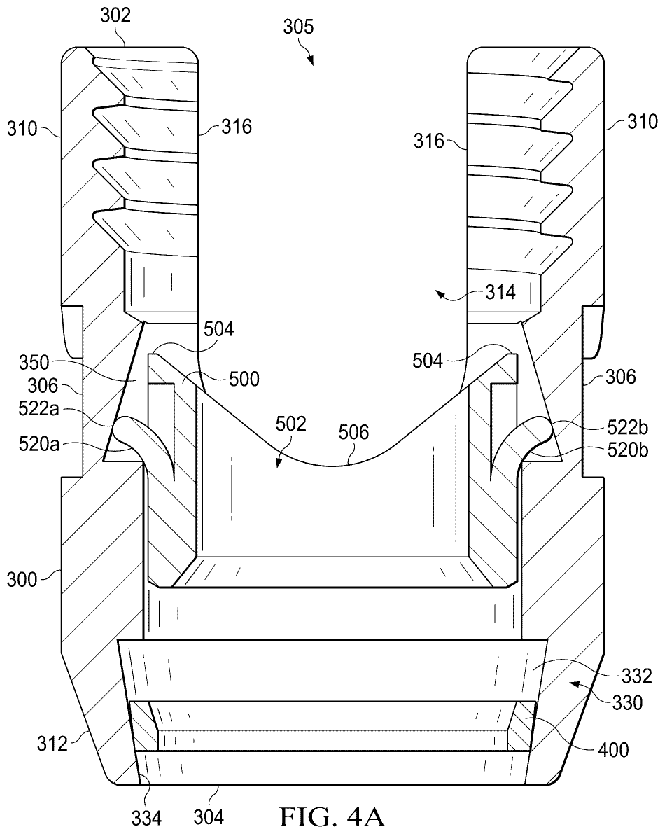

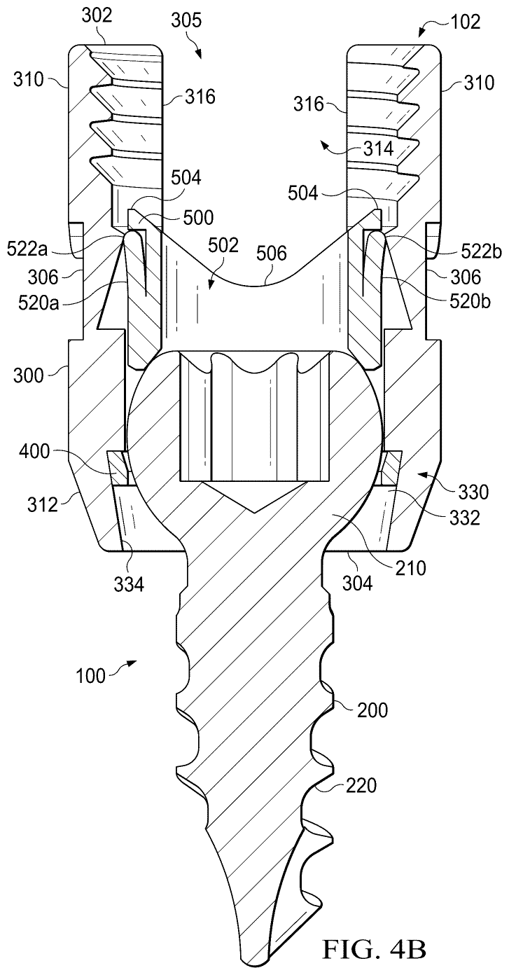

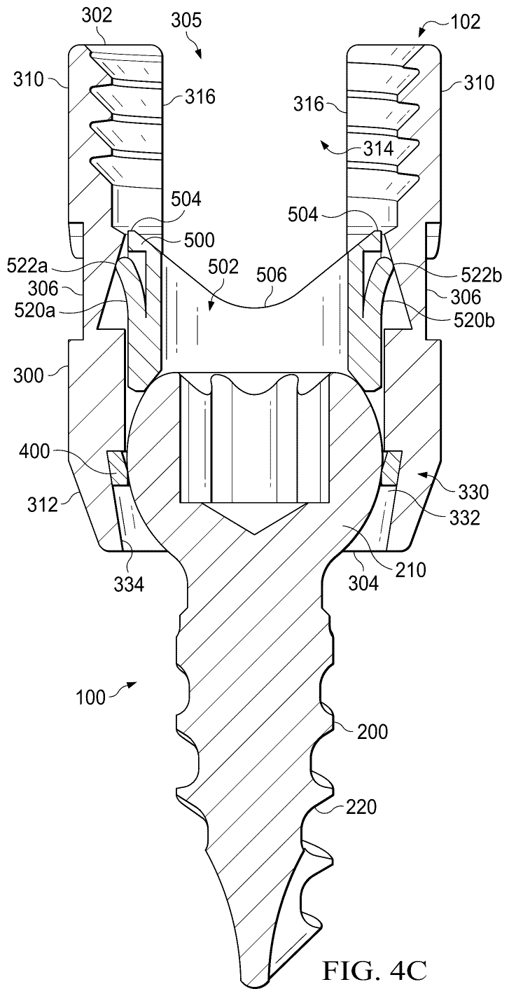

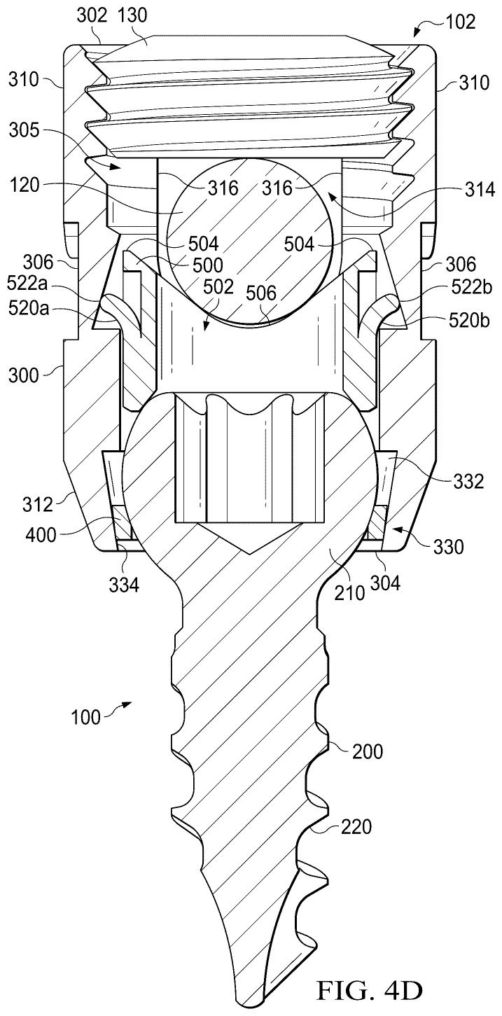

The body 300 further comprises a tapered recess 350 formed in an interior surface of the body. In the illustrated embodiment, the tapered recess includes two portions on either side of a U-shaped slot 314 of the body 300, in which the connecting rod is received. However, in other aspects, the receiver 102 comprises non-conical surfaces and shapes, such as a planar ramp surface. The recess 350 will be described in more detail with respect to FIGS. 4A-4D.

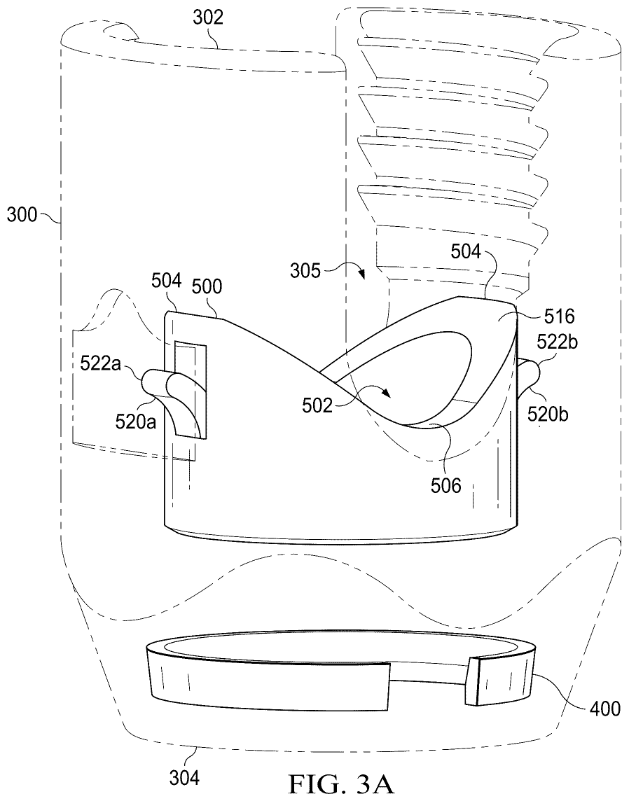

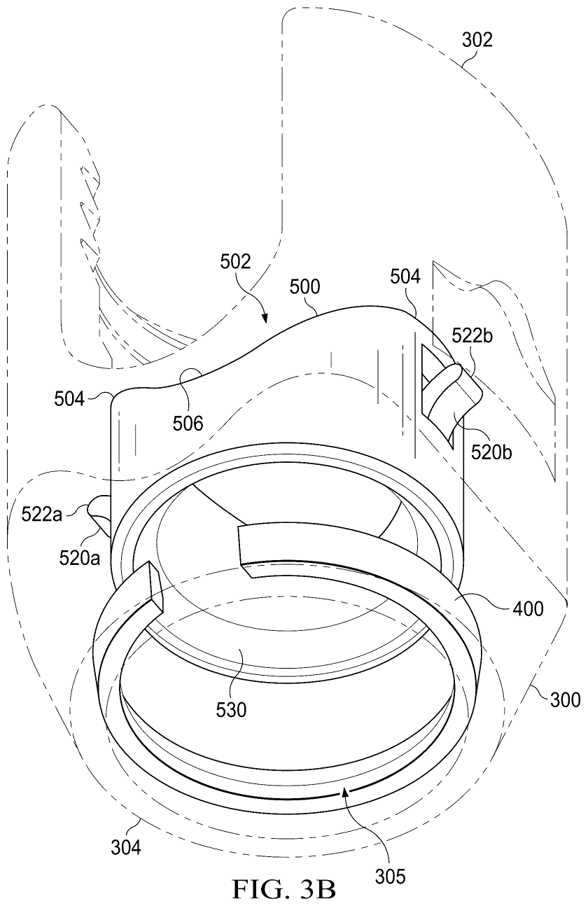

The receiver 102 also includes a pressure cap 500, which may also be referred to as a pressure member. The pressure cap 500 includes a body 510 defining a concave upper surface or top surface 516 for receiving the connecting rod, as described above. The pressure cap 500 may be saddle-shaped, meaning the pressure cap 500 has two ends 504 with an arched surface forming a depression 506 between the two ends 504. This saddle-shape may generally match the shape of the U-shaped slot 314 formed between the arms 310 of the body 300. Thus, the pressure cap 500 may be shaped to accept a rod that is placed within the U-shaped slot 314 of the body 300. The pressure cap 500 may also include a concave surface (530, FIG. 3B) on the bottom side of the pressure cap 500 to contact and engage a top surface of a screw head. However, in other embodiments, the pressure cap 500 may have any appropriate shape having a top for seating a rod and a bottom for contacting a screw head. For example, the pressure cap 500 may include a v-shaped depression, a rectangular depression, an elliptical depression, a hexagonal depression, and/or any other suitable shape for receiving the connecting rod. Similarly, the bottom surface of the pressure cap 500 may be flat, inclined, saddle-shaped and may be shaped elliptically, rectangularly, hexagonally or any other suitable shape for contacting and engaging a top surface of a screw head.

Moreover, the pressure cap 500 has an opening 502 extending through the center and aligning with the axial bore 305 of the body 300. The opening 502 allows an instrument to access a head of a screw when it is inserted into the receiver 102. For example, an interfacing portion or bit of a screw driver may be able to pass through the opening 502 of the pressure cap 500 so that the bone screw may be screwed into bone.

The pressure cap 500 includes a pair of resilient tabs 520a, 520b extending upward and outward from an outer surface of the pressure cap 500. The tabs 520a, 520b may alternatively be referred to as wings, projections, leaf springs, or any other suitable term. The resilient tabs 520a, 520b may be integrally formed with the pressure cap 500, or may comprise separate components or elements that are attached, fixed, or otherwise connected to the body of the pressure cap 500. For instance, the resilient tabs 520a and 520b may be adhered, welded, or press fit into a corresponding recess in the body of the pressure cap 500. In another example, the resilient tabs 520a and 520b may be machined from a monolithic or integral structure that forms the body of the pressure cap 500. The resilient tabs 520a and 520b may be formed by a combination of machining and permanent plastic deformation.

The resilient tabs 520a and 520b are shown in their relaxed state in FIG. 2B. In their relaxed state, the resilient tabs 520a and 520b protrude outward and upward along an arcuate path. The resilient tabs may be configured to flex inward by application of a force such that the tabs 520a, 520b elastically deform in a spring-like fashion. As will be explained in more detail below, the resilient tabs 520a and 520b are sized, shaped, and otherwise structurally configured to fit at least partially within and engage the tapered recess 350. The size and geometry of the resilient tabs 520a and 520b is such that the resilient tabs 520a and 520b contact and interfere with the sloped or tapered surface of the tapered recess 350 while permitting some longitudinal movement of the pressure cap 500 relative to the body 300. As the pressure cap 500 moves longitudinally upward, or approximately, relative to the receiver body 300, the tapered surfaces of the tapered recess 350 cause the resilient tabs 520a and 520b to flex inward. In other words, applying an upward or proximal force to the pressure cap 500 that overcomes the spring force of the resilient tabs 520a and 520b causes the pressure cap 500 to move upward or proximally relative to the body 300.

When the upward or proximal force is removed, the pressure cap 500 tends downward or distally as the resilient tabs 520a and 520b return to a relaxed state. More space is provided at or near the bottom of the tapered recess 350 for the resilient tabs 520a, 520b to relax and expand. Each of the resilient tabs 520a and 520b includes a respective rounded end 522a, 522b, which facilitate a sliding contact or engagement between the resilient tabs 520a, 520b, and the sloped surface of the tapered recess 350. The resilient tabs 520a, 520b project outward and into the tapered recess 350 to a sufficient degree that the rounded ends 522a and 522b maintain contact with the surface of the tapered recess 350 along a majority of the longitudinal travel of the pressure cap 500. The engagement between the resilient tabs 520a and 520b and the tapered recess 350 biases the pressure cap 500 downward to facilitate a friction fit or interference fit between the head of a bone screw and the receiver assembly 102. This friction fit provides sufficient friction to maintain a position and orientation of the receiver 102 relative to the bone screw but allows the physician to manually adjust the position and orientation of the receiver 102 relative to the bone screw prior to locking. It will be understood that the frictional relationship between the receiver 102 and the head of the bone screw, which is facilitated by the resilient tabs 520a, 520b of the pressure cap 500 and the tapered recess 350 of the body 300, may be described as a clutch mechanism, a friction fit mechanism, an interference fit mechanism, a friction hold mechanism, or any other suitable term. For the purposes of the present disclosure, the friction fit engagement between the head of the bone screw and the receiver 102 may contrast with locking of the assembly 100, in which a compression screw or set screw is urged down against the connecting rod to impart a much greater frictional force between the head of the bone screw and the retention ring and thereby lock the assembly 100 to prevent movement of the receiver 102 relative to the bone screw.

In some aspects the angle of the ramped or sloped surface of the tapered recess 800 forms an angle that is equal to or less than 85 degrees relative to the plane that is orthogonal to the longitudinal axis of the receiver 102. In another aspect, the angle is equal to or less than 81 degrees relative to the orthogonal plane. For instance, the angle may be 81 degrees, 80 degrees, 75 degrees, 70 degrees, 68 degrees, 65 degrees, or any other suitable angle.

FIGS. 3A and 3B show perspective views of the receiver 102 with the body 300 shown as being transparent for illustrative purposes. FIG. 3A shows a perspective side view of the receiver 102 and FIG. 3B shows a perspective bottom view of the receiver 102. When the receiver 102 is assembled, the retainer ring 400 is located around the axial bore 305 proximate the bottom 304 of the body 300. In this embodiment, the retainer ring 400 is a split ring that has a discontinuous annular shape configured to expand and/or retract to enlarge and/or reduce an inner diameter of the retainer ring 400. In other embodiments, the retainer ring 400 may be a continuous ring capable of expanding over a screw head when it is inserted from the bottom 304 of the body 300. The retainer ring 400 may be configured to lock the screw 200 into the retainer 102 once the screw head 210 has been inserted through a bottom opening of the retainer ring 400, as shown in FIGS. 4B-4D, for example. In some embodiments, the upper surface of the screw head 210 may be spherical, rounded, tapered, or otherwise configured to cause the retainer ring 400 to expand as the screw head 210 is pressed against the retainer ring 400 to allow the screw head 210 to pass through the retainer ring 400. Once the screw head 210 has passed through the retainer ring 400, the retainer ring 400 may relax and contract to lock against a bottom curved surface of the screw head 200. In some embodiments, an inner surface of the retainer ring 400 includes a ridge or seating feature configured to engage the bottom surface of the screw head 210. However, in other embodiments, the receiver 102 may have any appropriate component that locks the screw head 210 into the receiver 102, such as spring-loaded ball bearings, yielding locking ridge, and/or any other suitable feature.

In the illustrated embodiment, the pressure cap 500 is at a lowermost (or distal-most) longitudinal position in its range of travel. In this position, the resilient tabs 520A and 520B are relaxed and protruding outward into the tapered recess 350 (FIGS. 2A-2B). The rounded ends 522A and 522B of the resilient tabs 520A and 520B are rounded from an underside to a topside of the resilient tabs 520a, 520b. The rounded ends 520a, 520b are also rounded about the longitudinal axis of the body 300. In an exemplary aspect, the shape of the rounded ends is such that it facilitates point contact with the surface of the tapered recess 350. In other aspects, the shape of the rounded ends 522a, 522b may match or correspond to the circular or elliptical cross-sectional shape of the tapered recess 350, which may facilitate line contact with the surface of the tapered recess 350. In some aspects, each of the rounded ends 522a, 522b may be described as including sections of a toroidal surface.

It will be understood that the ends 522a, 522b may be rounded in other ways different from what is illustrated in FIGS. 2A and 2B. For instance, the rounding of the ends 522a 522b may comprise a partial cylindrical surface, a partial spherical surface, a planar surface, or any other suitable shape. The shape of the ends 522a, 522b, surface finish, and surface area of contact may be configured to reduce a chance of catching or binding. In this way, the tabs 520a, 520b may glide along the surface of the tapered recess 350 while compressing inward or relaxing outward.

As illustrated in FIGS. 4A-4D, the receiver 102 may allow for bottom-loading of the screw 200 through the bottom 304 of the axial bore 305 of the body 300. FIG. 4A shows a cross-sectional view of the receiver 102 through the arms 310 (according to the line shown in FIG. 2A). FIGS. 4B-4D show the same cross-sectional view as FIG. 4A at different stages of assembly of the pedicle screw assembly 100 (the receiver 102 with a screw 200 inserted). In some embodiments, the receiver 102 may be configured for assembly before or during a surgical procedure. For example, the physician may select the screw 200 based on the patient's anatomy and indications. In some embodiments, the screw 200 may be selected after the surgery has begun and after the surgeon has created an access through the patient's tissue to the bone. In other instances, the physician and/or surgeon may select the screw 200 before the surgery based on medical images of the patient's anatomy (e.g., x-ray, computed tomography, magnetic resonance imaging).

In some aspects, a physician may load the screw 200 into the receiver 102 to form a pedicle screw assembly 100 prior to inserting and driving the screws 200 into the patient's bone. The bottom-loading style of the assembly may be referred to as a modular assembly. The bottom-loaded modular assembly may be advantageous, in some aspects. For example, the modular assembly style of the receivers 102 may allow for the physician to choose a type and/or size of screw and assemble the receiver 102 and screw 200 during a spinal fixation procedure, based on the patient's anatomy and indications. The modular style may also allow for quick and efficient assembly with little or no disassembly of the receiver 102. In another aspect, the screw 200 may be inserted into the bone without the receiver 102 connected. The receiver 102 may then be installed over or “popped on” to the head of the bone screw once the screw is in place.

The upper surface of the screw head 210 may include a spherical, aspherical, or otherwise curved shape configured to engage the bottom surface of the pressure cap 500. In other embodiments, the screw head 210 may include a conic section shape. Accordingly, the screw head 210 may be curved about at least one axis to allow the screw head 210 to continuously rotate relative to the pressure cap 500. In other embodiments, the screw head 210 may include a polygonal shape having a plurality of flat surfaces arranged around an axis of the screw 200. For example, the screw head 210 may include, on the upper surface, 10, 20, 25, 30, or any other suitable number of flat surfaces arranged around the axis of the screw 200.

The screw 200 includes a distal threaded shaft 220 comprising screw threads configured to drive into and engage the patient's bone. In the illustrated embodiment, the threads are right-handed threads. In other embodiments, the threads may be left-handed threads. The threads may have any suitable pitch, depth, and/or other geometric characteristics based on the target bone or tissue and application for the assembly. The screw 200 may be machined, laser sintered, 3D printed, or otherwise manufactured by any suitable manufacturing process. It will be understood that the threaded portion of the shaft of the screw 200 may extend a greater or lesser portion of the shaft than what is shown in FIGS. 4B-4D.

In FIG. 4A, the receiver 102 is shown prior to inserting the bone screw. In this embodiment, the retainer ring 400 is disposed within a retainer ring recess 330 within the base 312 of the body 300. The retainer ring recess 330 allows the retainer ring 400 sufficient room to expand over the screw head 210. The retainer ring recess 330 is relatively wider at the top than at the bottom. Thus, as the screw head 210 is pushed upward, the retainer ring 400 moves up and expands outward, allowing the screw head 210 to pass through the retainer ring 400. In the illustrated embodiment, the retainer ring recess 330 may be described as having an upper portion 332 and a lower portion 334, though the portions 332, 334 may be parts of a single recess having a continuous surface surrounding the recess 330. In this way, the recess 330 has relatively wider diameters in the upper portion 332 than in the lower portion 334. The recess comprises a conical shape, though other types of shapes and recesses may be provided, including spherical, parabolic, and/or any other suitable shape. The lower portion 334 is provided and shaped to allow sufficient room for the screw head 210 to pass through, and the retainer ring 400 can expand in upper portion 332, as explained further below. The retainer ring 400 may then be pressed upward into the upper portion 332 as the screw head 210 is inserted through the bottom of the body 300 of the receiver 102. As the retainer ring 400 moves into the upper portion 332, the retainer ring 400 may expand more than it expands in the lower portion 334. When the screw head 210 is fully seated, the retainer ring 400 may be disposed between the upper 332 and lower 334 portions or in the upper portion 332. In some embodiments, when the screw head 210 is fully seated, the retainer ring 400 rests on a lower shoulder in the retainer ring recess 330. In some embodiments, when the screw head 210 is fully seated, the retainer ring 400 may not be disposed in the lower portion 334. The upper portion 332 and/or lower portion 334 may be tapered so that the upper portions 332 and/or lower portion 334 are wider at the top than at the bottom. However, in other embodiments, may have a different shape. In some embodiments, the retainer ring recess 300 does not have distinct upper 332 and lower 334 portions, but tapers from the top to the bottom. In some embodiments, the retainer ring recess 330 may not allow the retainer ring 400 to move up and down and may only allow the retainer ring 400 to expand outward. In other embodiments, the body 300 may not have a retainer ring recess 330 and instead may be attached to the wall of the axial bore 305 through another appropriate method.

The pressure cap 500 is shown in its lower-most or distal-most position in FIG. 4A. This position may be referred to as its relaxed position or default position. In this relaxed position, the resilient tabs 520a, 520b extend into lower portions of the recess 350. The tabs 520a, 520b may remain partially compressed or flexed inward by the wall of the recess 350 in the relaxed position. In other embodiments, the tabs 520a, 520b may be loose in the relaxed position shown in FIG. 4A.

The receiver 102 may be assembled by placing the retainer ring 400 in the retainer ring recess 330. The retainer ring 400 may sit in the retainer ring recess 330 or may be affixed to the retainer ring recess 330 using, for example, an adhesive. The retainer ring 400 may be inserted through either the top 302 or bottom 304 of the axial bore 305. Moreover, the pressure cap 500 may be inserted into the axial bore 305, or through the bottom opening 304. The receiver 102 may be assembled in any order.

The pedicle screw assembly 100 may be further assembled by inserting the screw 200 through the bottom opening 304 of the receiver 102 until it is locked within the receiver 102. FIGS. 4B-4D show the pedicle screw assembly 100 as it is being assembled. FIG. 4B shows a cross section of the pedicle screw assembly 100 as the screw 200 is being pushed upwards through the bottom opening 304 of the receiver 102. As shown in FIG. 4A, before the screw head 210 is inserted, the retainer ring 400 may be disposed between the upper 332 and lower 334 portions of the retainer ring recess 330. As the screw head 210 is pushed upwards, as shown in FIG. 4B, the retainer ring 400 is pushed upward into the wider upper portion 332 of the retainer ring recess 330 and expands around the screw head 210. While the screw head 210 is pushed upwards, the screw head 210 also contacts the lower concave surface of the pressure cap 500, pushing the pressure cap upwards. The upward movement of the pressure cap causes the tabs 520a, 520b to deflect inward to a compressed state, creating a spring force between the screw head 210 and the pressure cap 500. In the compressed and raised state, the tabs 520a, 520b are disposed in an upper portion of the tapered recess 350, which may have a smaller diameter or width that the lower portion of the tapered recess 350.

FIG. 4C shows the pedicle screw assembly 100 after the screw head 210 has been inserted through the retainer ring 400 and is returning to a seated position. As described above, the retainer ring recess 330 may be wider at the top than at the bottom, giving the retainer ring 400 additional room to expand when the retainer ring 400 is pushed to the top of the retainer ring recess 330 so that the screw head 210 can pass through the expanded retainer ring 400. Once the retainer ring 400 has passed over the widest part of the screw head 210, the retainer ring 400 may contract, locking the screw head 210 into the receiver 102. In some embodiments, the retainer ring 400 may contract to a width larger than the width of the lower portion 334 of the retainer ring recess 330. The pressure cap 500 is retreating from its uppermost position shown in FIG. 4B, and its resilient tabs 520a, 520b are opening or flexing outward accordingly as there is greater room to expand within the tapered recess 350.

The respective tapers of the recesses 350, 330 are oriented in opposite directions, such that the recess 330 is widest at the top of the recess 330, and the tapered recess 350 is widest at the bottom of the tapered recess 350. The recesses 330, 350 are shown as separated by a section having a smaller inner diameter than the tapered recess 350 at its widest (most distal) end. In other embodiments, the recesses 330, 350 may not be separated by a narrow section. For instance, the widest portion of the recess 330 may have a smooth transition to the widest portion of the recess 350.

FIG. 4D shows the pedicle screw assembly 100 after assembly. After the screw 200 is pressed upward through the bottom 304 of the receiver 102, the upward pressure on the screw 200 can be removed, thus allowing the screw 200 to settle into an assembled configuration. The bottom of the screw head 210 rests within the base 312 of the body 300. In some embodiments, as the retainer ring 400 contracts, the retainer ring 400 may move downward in the retainer ring recess 330. The screw head 210 may also press downward on the retainer ring 400 as it moves downward within the receiver 102. When the screw head 210 is seated within the receiver 102, the retainer ring 400 may be disposed between the upper 332 and lower 334 portions of the retainer ring recess 330. The bottom of the pressure cap 500 maintains contact with the top of the screw head 210, and the tabs 520a, 520b remain at least partially compressed or flexed such that the pressure cap 500 continues to impart a downward force on the screw head 210 even while the screw head 210 is seated on the retainer ring 400. This, in turn, creates a frictional force between the screw head 210 and the interior surface of the retainer ring 400. The frictional force is sufficient to maintain a position of the receiver 102 relative to the screw 200 as explained above. This friction fit or interference fit allows the physician to manually manipulate the position and/or orientation of the receiver 102 relative to the screw 200 such that the receiver 102 maintains its position and orientation so that the connecting rod can be inserted prior to locking the assembly.

The materials of the receiver 102 may be biocompatible, and may have other structural characteristics appropriate for use in spinal fixation. For example, the body 300, pressure cap 500, pin 320, retainer ring 400, and/or the screw 200 may include a biocompatible metal, such as stainless steel, titanium, and/or alloys thereof. In other embodiments, one or more components of the receiver 102 may include a polymer material, such as DELRIN, polyether ether ketone (PEEK), polytetrafluoroethylene (PTFE), polysulfone (PS), polycarbonate, and/or any other suitable polymeric material. One or more components of the receiver 102 may be manufactured by milling, machining, casting, molding, laser sintering, 3D printing, and/or any other suitable process. The components of the receiver 102 may be formed of the same materials or of different materials.

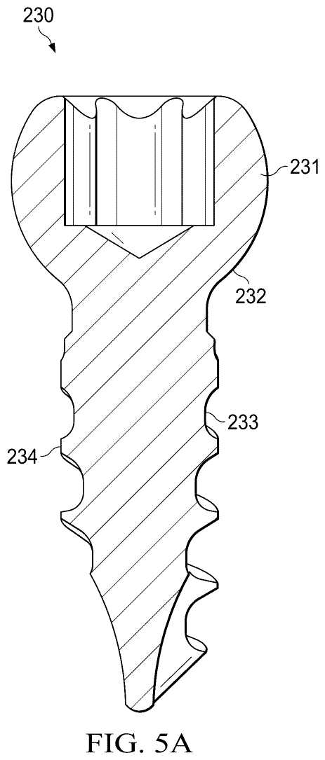

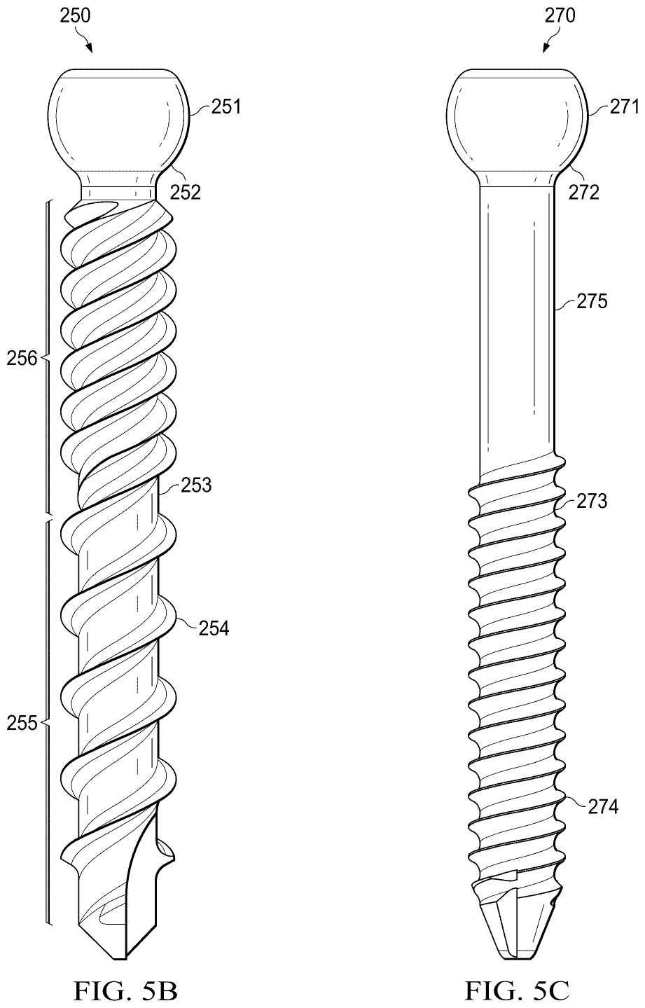

FIGS. 5A-5C show various embodiments of a screw 200 for use in the pedicle screw assembly 100. FIG. 5A illustrates a dual lead pitch screw 230 as shown in FIGS. 1 and 4B-4D. In the illustrated embodiment, the dual lead pitch screw 230 comprises a head 231 having a spherical-shaped bottom 232 and a shaft 233. The shaft 233 comprises threading 234 that extends along the length of the shaft 233. The threading 234 of the dual lead pitch screw comprises two starts. The dual lead pitch screw 230 may be advantageous for use in cortical bone. However, in other embodiments, the screw 250 may not be dual lead and instead may have one start or more than two starts. Moreover, the screw 250 may have any appropriate pitch or lead.

FIG. 5B illustrates a part dual lead, part single lead screw 250. This embodiment comprises a head 251 having a spherical-shaped bottom 252 and a shaft 253. The shaft 253 comprises a threading 254 that extends completely or partially down the length of the shaft 253. Unlike the embodiment illustrated in FIG. 5A, the threading 254 in the embodiment illustrated in FIG. 5B comprises a first portion 255 that has one start and a second portion 256 that has two starts. The first portion 255 and the second portion 256 may be any appropriate length. This embodiment may be advantageous for use in bone that comprises a cortical layer and a cancellous layer. In other embodiments, the threading may comprise any appropriate number of portions with any appropriate number of starts. Moreover, the screw 250 may have any appropriate pitch or lead.

FIG. 5C illustrates a smooth shank screw 270. This embodiment comprises a head 271 having a spherical-shaped bottom 272 and a shaft 273. In this embodiment, the shaft 273 comprises threading 274. The threading 274 in screw 270 illustrated in FIG. 5C has threading 274 that extends partially along the length of the shaft 273. Thus in the embodiment of FIG. 5C, the screw 270 comprises a smooth portion 275 between the threading 274 and the head 271. The length of the smooth portion 275 may be any appropriate length. Moreover, the screw 270 may comprise any appropriate number of starts and may comprise multiple portions with different numbers of starts.

Any of the screws described herein may be any appropriate length. For example, the screws may be 20 mm, 25 mm, 30 mm, 35 mm, 40 mm, 45 mm, 50 mm, 55 mm, 60 mm, or any other length.

The receiver 102 of the pedicle screw system 100 may be compatible with any of the screws 230, 250, 270 shown in FIGS. 5A, 5B, 5C, respectively, or any other appropriate screw according to the embodiments contemplated by the present disclosure. Thus, the same receiver 102 may be used for any appropriate screw 200. This allows the physician to have one type of receiver 102 but choose the desired screw 200 during the procedure. In other aspects, the receiver 102 may be specifically sized, shaped, or otherwise configured for use for one type of screw 200 but not for a different type of screw.

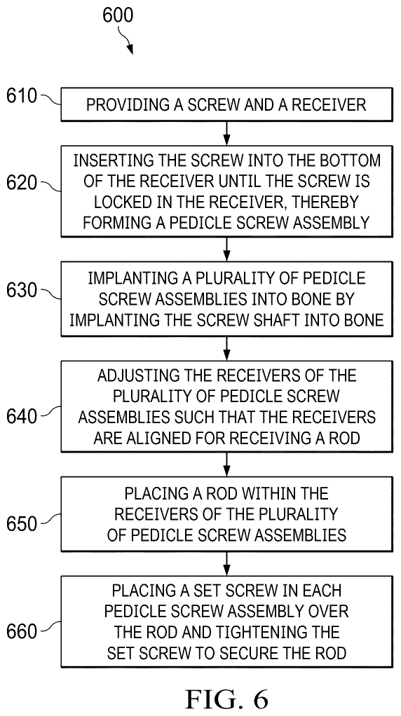

FIG. 6 shows a method 600 of treating a spinal condition using the pedicle screw assembly 100. Step 610 of the method 600 includes providing a screw 200 and a receiver 102. The receiver 102 may be any receiver 102 described herein, including any of the receivers 102 or parts of receivers 102 shown in FIGS. 1-5C. The screw 200 may be any screw 200 described herein, including the screws 200 shown in FIGS. 1, 4B-4D and 5A-5C. The surgeon or technician may select a receiver and fastener assembly from a plurality of receiver and fastener assemblies based on the patient's anatomy and/or indications. Step 620 of the method includes inserting the screw 200 into the bottom 304 of the receiver 102 until the screw 200 is locked in the receiver 102, thereby forming a pedicle screw assembly 100. As the screw 200 moves upward through the receiver 102, the retainer ring 400 may expand over the head 210 of the screw, the pressure cap 500 may be pressed upward, and the resilient tabs 520a, 520b may be deformed as described in reference to FIG. 4B. When the upward force is removed from the screw 200, the screw 200 may move downward slightly to rest in the base 312 of the body 300. As the screw 200 moves downward, the retainer ring 400 may move downward, the pressure cap 500 may move downward and the resilient tabs 520a, 520b may expand as described in reference to FIGS. 4C and 4D. However, the screw 200 may be inserted into and be locked in the receiver 102 in any way as described herein.

Step 630 of the method 600 includes implanting a plurality of pedicle screw assemblies 100 into bone by implanting the screw shaft 220 into the bone. In some embodiments, the bone may be a vertebrae 110. As described herein, an instrument may releasably engage the engagement feature 306 of the receiver 102. The instrument may then be used to position the pedicle screw assembly 100 at the desired position. The same instrument or a different instrument may drive the screw 200 into the bone. For example, a screw driver may pass through the opening 502 of the pressure cap 500 to access the screw head 210. The screw driver may then be used to screw the screw shaft 220 into the bone. However, the screw 200 may be driven into the bone using any appropriate method. Step 640 of the method 600 includes adjusting the receivers 102 of the plurality of pedicle screws 100 such that the receivers 102 are aligned for receiving a rod 120. An instrument may engage with the engagement feature 306 of the receiver 102 to move the receiver 102 independent of the screw 200. The receiver 102 may be moved into any appropriate position for receiving a rod. The frictional force that the pressure cap 500 applies to the screw head 210 may retain the position and orientation of the receiver 102 relative to the screw head 210.

Step 650 of the method 600 includes placing a rod 120 within the receivers 102 of the plurality of pedicle screw assemblies 100. Once the receivers 102 are aligned, a rod 120 may be placed such that it fits within the U-shaped slot 314 formed by the arms 310 of the receiver 102. The rod 120 may be bent or curved into the desired shape before or while placing the rod 120 into the receiver 102. Step 660 of the method 600 includes placing a set screw 130 in each pedicle screw assembly 100 over the rod 120 and tightening the set screws 130 to secure the rod 120. Tightening the set screws 130 may also secure the position and orientation of the receivers 102 relative to the screws 200. The set screws 130 may be any appropriate set screw 130 design, including the design shown in FIG. 1. The set screw 130 may have threads that engage the threads 316 of the arms 310 of the body 300. An instrument, such as, for example, a screwdriver, may be used to tighten the set screw 130 until it contacts and presses against the rod 120. The set screws 130 may secure the rod 120 and/or the receivers such that they do not move. In some embodiments, the rod 120 may be tightened such that it stabilizes the vertebrae 110.

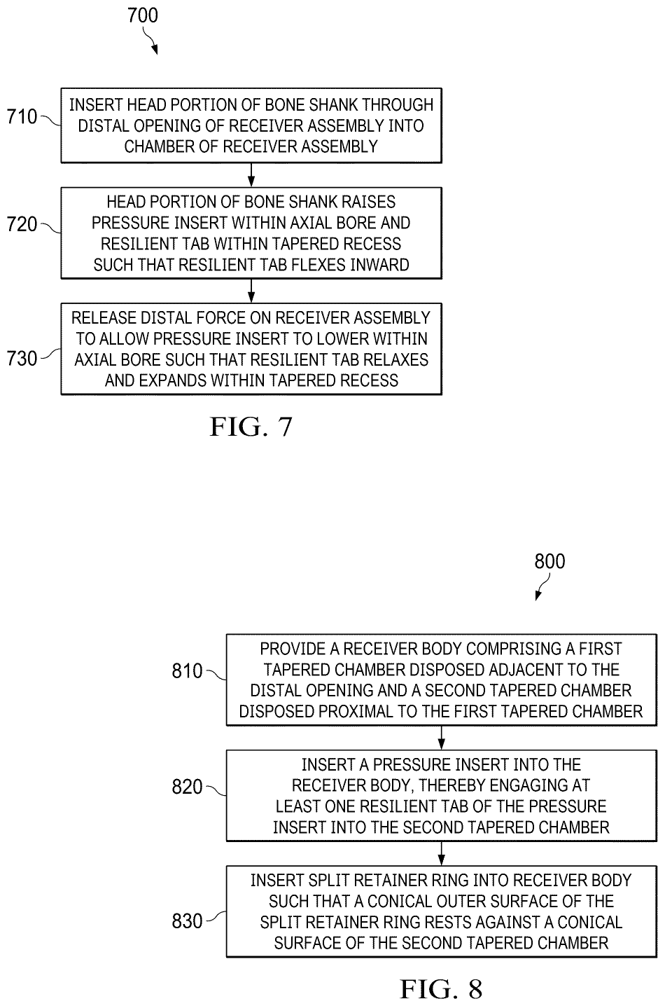

FIG. 7 shows a method 700 of assembling a spinal screw assembly. It will be understood that the method 700 may be used to assemble any of the screw assemblies described herein, including those illustrated in FIGS. 1-4D. At step 710, the physician inserts a head portion of a bone shank through a distal opening of a receiver assembly and into a chamber of the receiver assembly. The receiver assembly comprises a receiver body, comprising: the distal opening; a conical interior surface disposed about the chamber; an axial bore extending longitudinally through the receiver body; and a tapered recess defined in a surface of the axial bore. The receiver further comprises a split retainer ring disposed in the chamber; and a pressure insert disposed at least partially within the axial bore. The pressure insert comprises a resilient tab projecting proximally and outward from an exterior surface of the pressure insert, the resilient tab comprising a rounded face engaging the tapered recess.

At step 720, the head portion of the bone shank raises the pressure insert within the axial bore, and a resilient tab of the pressure insert engaged within a tapered recess of the receiver assembly also moves upward or proximally such that the resilient tab flexes upward. By inserting the head portion of the bone shank through the distal opening, the head portion pushes through the distal opening of the receiver body and through the split retainer ring to cause the split retainer ring to elastically expand about the head portion, and thereafter to collapse about a neck of the bone shank to retain the head portion within the chamber in a pivotable relationship with the receiver body. Further, inserting the head portion causes the pressure insert to move to a first longitudinal position;

At step 730, the physician releases a distal force on the receiver assembly to allow the receiver assembly to return, or begin to return, to a relaxed state. In the relaxed state, the resilient tab expands within the tapered recess and slides distally while the resilient tab maintains contact with a surface within the tapered recess.

FIG. 8 shows a method 800 of assembling a receiver assembly. It will be understood that the method 800 may be used to assemble any of the receiver assemblies described herein, including those illustrated in FIGS. 1-4D. At step 810, the physician provides a receiver body. The receiver body comprises: an axial bore extending longitudinally through the receiver body from a proximal opening of the receiver body to a distal opening, the distal opening comprising a width; a first tapered chamber disposed adjacent to the distal opening, wherein the first tapered chamber increases in diameter toward a top portion of the first tapered chamber; and a second tapered chamber disposed proximal to the first tapered chamber, wherein the second tapered chamber increases in diameter toward a bottom portion of the second tapered chamber.

At step 820, the physician inserts a pressure insert into the receiver body through one of the proximal opening or the distal opening. Inserting the pressure insert may include engaging at least one resilient tab of the pressure insert into the second tapered chamber. As explained above, in an exemplary embodiment, the pressure insert includes two resilient tabs on opposing sides of the pressure insert. The second tapered chamber may include a conical bore. In other embodiments, the second tapered chamber includes two conical bore sections. In other embodiments, the second tapered chamber comprises a recess into the sidewall of the axial bore that includes a planar ramped or angled surface.

At step 830, the physician inserts a split retainer ring through the distal opening such that a conical outer surface of the split retainer ring rests against a conical surface of the second tapered chamber. In some aspects, step 830 comprises compressing the split retainer ring from a first width to a second width, wherein the second width of the split retainer ring is smaller than the width of the distal opening.

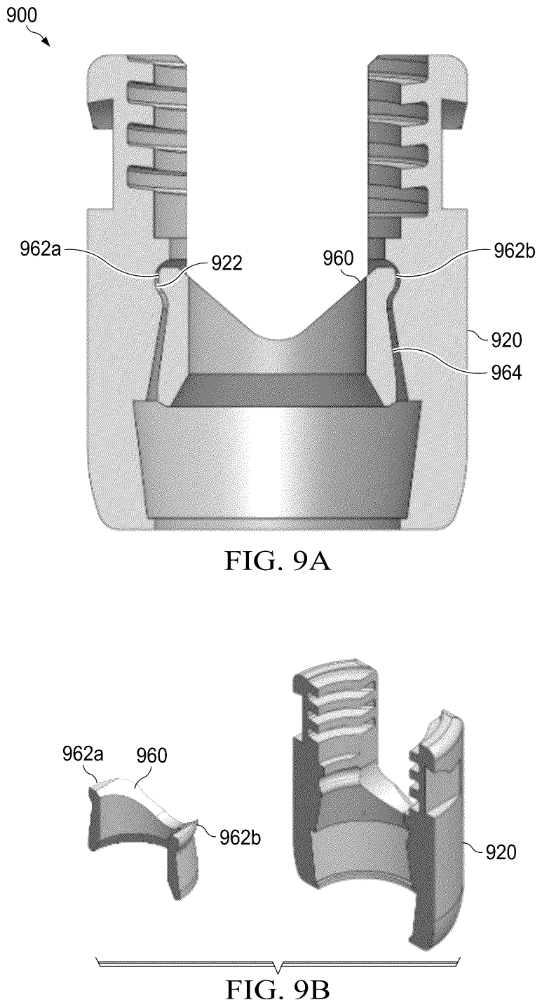

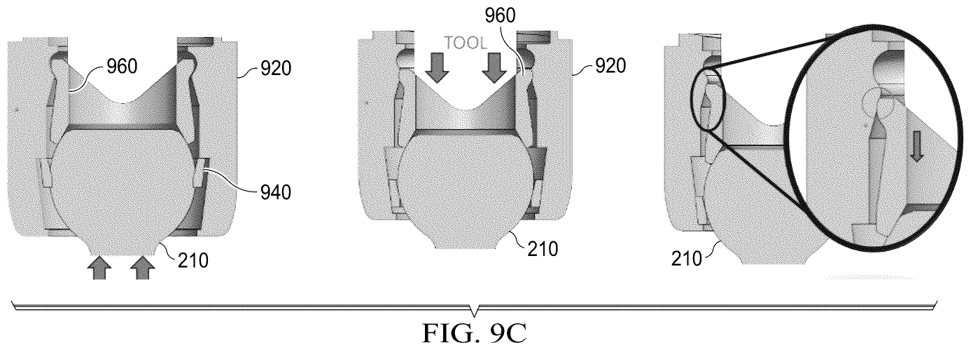

FIGS. 9A-9C illustrate other embodiments of a receiver assembly 900, according to some aspects of the present disclosure. FIG. 9A is a cross-sectional view of the receiver 900 according to a first embodiment, and FIG. 9B is a perspective cross-sectional view of the two components of the receiver 900. FIG. 9C includes three cross-section views of the receiver 900 according to a second embodiment during three stages of assembly. Receiver 900 may be similar to receiver 102 illustrated in FIGS. 1-4D in that the receiver 900 comprises a body 920, a retainer ring 940 (FIG. 9C), and a pressure cap 960. However, in the receiver 900 shown in FIGS. 9A-9C, the resilient tabs 962a, 962b protrude from the pressure cap in a different way than those in FIGS. 4A-4D. Further, the outer profile of the body of the pressure cap 960 comprises a conical section 964. The conical profile may provide a relief whereby the arms or tabs of the pressure cap 960 can move into and out of the corresponding recess 922 in the body 920. Further, the recess 922 that receives the tabs 962 is not conical as in some of the other embodiments described herein.

In the illustrated embodiment of FIGS. 9A-9C, the recess 922 includes a rounded groove the provides less travel than the tapered recesses illustrated and described above. The recess 922 in the embodiment of FIGS. 9A-9C and the tabs 962 of the pressure insert 960 cooperate similar to a detent. In this way, the upper sidewalls of the pressure insert 960 may flex inward to allow the tabs 962 to enter and expand within the recess 922 when the pressure insert is inserted into the receiver body 920 from the distal opening. In this state, the pressure insert 960 is supported in a raised position. In some aspects, the pressure insert 960 may be lifted into this position (shown in FIG. 9A), when the head of the bone screw is inserted into the receiver assembly through the distal opening. FIG. 9C illustrates the process whereby the pressure insert 960 is urged upward such that the tabs 962 engage the recess 922 and the split retainer ring expands around and over the head of the bone screw. Next, within the bone screw head 210 inserted into the receiver's chamber, the physician may use a tool to urge the pressure insert downward with sufficient force such that the resilient tabs 962 of the pressure insert 960 are urged inward due to the engagement of the rounded tabs with the rounded bottom of the recess 922. Then, once the resilient tabs 962 disengage with the recess 922, they then engage a lower tapered surface. As shown in FIG. 9C, the interaction between the resilient tabs and the tapered surface is similar in at least some aspects to the interaction between the resilient tabs and the tapered surface of the tapered recess 350 shown in FIGS. 4A-4D.

It will be understood that one or more embodiments described above may be modified in one or more ways without departing from the scope of the present disclosure. In some embodiments, a body may include fewer or more engagement features than the two engagement features shown above. In some embodiments, a receiver may not allow for modular assembly. For example, a receiver may not include the retainer ring illustrated above. In this regard, an implantable assembly may be configured such that a bottom surface of the screw head directly contacts a seating surface of the body. In some embodiments, the pressure cap may have a pin that fits into a slot of the body instead of the body having a pin that fits into the slot of the pressure cap as shown above.

Aspects, components, and features described above may be used in a variety of skeletal stabilization and/or fixation systems. Further, although embodiments of the present disclosure may be described as spinal implants or spinal fixation devices, it will be understood that the devices described above may be used for a variety of skeletal stabilization and/or fixation procedures.

Persons of ordinary skill in the art will appreciate that the implementations encompassed by the present disclosure are not limited to the particular exemplary implementations described above. In that regard, although illustrative implementations have been shown and described, a wide range of modification, change, combination, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A fastener assembly for a spinal fixation system, comprising:
a bone shank comprising a head portion and a distal threaded portion configured to be implanted into bone;
a receiver comprising:
an axial bore extending longitudinally through the receiver from a proximal opening of the receiver to a distal opening; and
an interior surface surrounding a portion of the axial bore, the interior surface defining a tapered recess, the tapered recess increasing in depth toward a distal end of the tapered recess;
a pressure insert disposed at least partly within the axial bore, the pressure insert comprising:
a saddle configured to seat a fixation rod;
a distally-facing concave surface configured to contact the head portion of the bone shank; and
a resilient tab projecting proximally and outward from an exterior cylindrical surface of the pressure insert;
wherein the pressure insert is longitudinally displaceable in the axial bore from a first longitudinal position to a second longitudinal position,
wherein longitudinal displacement of the pressure insert to the second longitudinal position is resisted by a spring force facilitated by the rounded face of the resilient tab engaging the tapered recess, and
wherein, when the pressure insert returns to the first longitudinal position, the spring force urges the pressure insert distally against the head portion of the bone shank to facilitate a friction-fit engagement between the head portion of the bone shank and the receiver.

2. The fastener assembly of claim 1, wherein:
the receiver further comprises a chamber disposed adjacent to the distal opening and configured to receive the head portion of the bone shank therein;
the fastener assembly further comprises a split retainer ring disposed in the chamber; and
the friction-fit engagement is at least partially between the head portion of the bone shank and the split retainer ring.

3. The fastener assembly of claim 2, wherein the head portion of the bone shank comprises a spherical surface, wherein the split retainer ring comprises an interior concave surface configured to contact the spherical surface to thereby provide a pivotable relationship between the head portion of the bone shank and the interior concave surface of the split retainer ring.

4. The fastener assembly of claim 3, wherein:
the receiver comprises an interior conical surface defining the chamber,
the split retainer ring further comprises an outer conical surface, and
when the pressure insert is in the second longitudinal position, the outer conical surface abuts the interior conical surface of the receiver.

5. The fastener assembly of claim 4, further comprising a compression screw configured to be threadably received into an upper opening of the receiver along a longitudinal axis of the receiver, and wherein the compression screw is configured to compress the fixation rod against the pressure insert, which in turn locks the fastener assembly by simultaneously urging:
the spherical surface of the head portion of the bone shank against the interior concave surface of the split retainer ring; and
the outer conical surface of the split retainer ring against the interior conical surface of the receiver.

6. The fastener assembly of claim 1, wherein the resilient tab comprises a rounded face contacting the tapered recess.

7. The fastener assembly of claim 6, wherein the rounded face is configured to make point contact with the tapered recess.

8. The fastener assembly of claim 1, further comprising a second resilient tab disposed in the tapered recess, wherein the tapered recess comprises a conical bore.

9. The fastener assembly of claim 1, wherein:
the tapered recess comprises a planar ramped surface configured to contact the resilient tab;
the interior surface surrounding the axial bore further defines a second tapered recess comprising a second planar ramped surface; and
wherein the pressure insert further comprises a second resilient tab disposed in the second tapered recess and configured to make sliding contact with the second planar ramped surface.

10. A receiver for a polyaxial bone screw assembly, the receiver comprising:
a body, comprising:
an axial bore extending longitudinally through the receiver from a proximal opening of the receiver to a distal opening;
a chamber disposed adjacent to the distal opening and configured to receive a head portion of a bone shank therein; and
an interior surface surrounding a portion of the axial bore, the interior surface defining a tapered recess, the tapered recess increasing in depth toward a distal end of the tapered recess;
a split retainer ring disposed in the chamber; and
a pressure insert disposed at least partly within the axial bore, the pressure insert comprising:
a first surface configured to seat a fixation rod;
a distally-facing concave surface configured to contact the head portion of the bone shank; and
a resilient tab projecting proximally and outward from an exterior cylindrical surface of the pressure insert, the resilient tab comprising a rounded face engaging the tapered recess,
wherein the pressure insert is longitudinally displaceable in the axial bore from a first longitudinal position to a second longitudinal position,
wherein longitudinal displacement of the pressure insert to the second longitudinal position causes the resilient tab engaging the tapered recess to deflect, thereby providing a spring resistance in a distal direction.

11. The receiver of claim 10, wherein the split retainer ring comprises an interior concave surface.

12. The receiver of claim 11, wherein the body further comprises a conical bore, wherein:
the split retainer ring further comprises an outer conical surface, and
when the pressure insert is in the second longitudinal position, the outer conical surface abuts the conical bore.

13. The receiver of claim 10, wherein the resilient tab comprises a rounded face contacting the tapered recess.

14. The receiver of claim 13, wherein the rounded face is configured to make point contact with the tapered recess.

15. The receiver of claim 10, further comprising a second resilient tab disposed in the tapered recess, wherein the tapered recess comprises a conical bore.

16. The receiver of claim 10, wherein:
the tapered recess comprises a planar ramped surface configured to contact the resilient tab;
the interior surface surrounding the axial bore further defines a second tapered recess comprising a second planar ramped surface; and
wherein the pressure insert further comprises a second resilient tab disposed in the second tapered recess and configured to make sliding contact with the second planar ramped surface.

17. A receiver body for a polyaxial bone screw assembly, the receiver body comprising:
an axial bore extending longitudinally through the receiver body from a proximal opening of the receiver body to a distal opening;
a first tapered chamber disposed adjacent to the distal opening and configured to receive a head portion of a bone shank therein, wherein the first tapered chamber increases in width toward a top portion of the first tapered chamber;
a second tapered chamber disposed proximal to the first tapered chamber, wherein the second tapered chamber increases in width toward a bottom portion of the second tapered chamber; and
a channel defined in an upper portion of the receiver body and configured to receive a fixation rod therein.

18. The receiver body of claim 17, wherein the first tapered chamber comprises a first conical bore.

19. The receiver body of claim 18, wherein the second tapered chamber comprises a second conical bore.

20. The receiver body of claim 18, wherein the second tapered chamber comprises a first planar ramped surface, and wherein the receiver body comprises a third tapered chamber comprising a second planar ramped surface.

21. A method for assembling a polyaxial fastener assembly, comprising:
inserting a head portion of a bone shank through a distal opening of a receiver assembly and into a chamber of the receiver assembly, wherein the receiver assembly comprises:
a receiver body, comprising:
the distal opening;
a conical interior surface disposed about the chamber;
an axial bore extending longitudinally through the receiver body; and
a tapered recess defined in a surface of the axial bore;
a split retainer ring disposed in the chamber; and
a pressure insert disposed at least partially within the axial bore, the pressure insert comprising:
a resilient tab projecting proximally and outward from an exterior surface of the pressure insert, the resilient tab comprising a rounded face engaging the tapered recess,
wherein the inserting comprises pushing the head portion through the distal opening of the receiver body and through the split retainer ring to:
cause the split retainer ring to elastically expand about the head portion, and thereafter to collapse about a neck of the bone shank to retain the head portion within the chamber in a pivotable relationship with the receiver body; and
cause the pressure insert to move to a first longitudinal position;
wherein the method further comprises:
releasing a distally-directed force on the receiver assembly to allow the pressure insert to return to a second longitudinal position, wherein, in the second longitudinal position, a spring force provided by the resilient tab urges the pressure insert distally against the head portion of the bone shank to facilitate a friction-fit engagement between the head portion of the bone shank and the split retainer ring.

22. A method for assembling a polyaxial fastener assembly, comprising:
providing a receiver body, comprising:
an axial bore extending longitudinally through the receiver body from a proximal opening of the receiver body to a distal opening, the distal opening comprising a width;
a first tapered chamber disposed adjacent to the distal opening, wherein the first tapered chamber increases in diameter toward a top portion of the first tapered chamber; and
a second tapered chamber disposed proximal to the first tapered chamber, wherein the second tapered chamber increases in diameter toward a bottom portion of the second tapered chamber;
inserting a pressure insert into the receiver body through one of the proximal opening or the distal opening, wherein the inserting the pressure insert comprises engaging at least one resilient tab of the pressure insert into the second tapered chamber; and
inserting a split retainer ring through the distal opening such that a conical outer surface of the split retainer ring rests against a conical surface of the second tapered chamber, wherein the inserting the split retainer ring comprises compressing the split retainer ring from a first width to a second width, wherein the second width of the split retainer ring is smaller than the width of the distal opening.

23. A fastener kit for a spinal fixation system, comprising:
a bone shank comprising a head portion and a distal threaded portion configured to be implanted into bone;
a receiver comprising:
an axial bore extending longitudinally through the receiver from a proximal opening of the receiver to a distal opening;
an interior surface surrounding a portion of the axial bore, the interior surface defining a tapered recess, the tapered recess increasing in depth toward a distal end of the tapered recess; and
a lower tapered chamber adjacent the distal opening;
a pressure insert configured to be positioned in the axial bore, the pressure insert comprising:
a saddle configured to seat a fixation rod;
a distally-facing concave surface; and
a resilient tab projecting proximally and outward from an exterior cylindrical surface of the pressure insert, wherein the resilient tab is sized and shaped to extend at least partially within the tapered recess of the receiver;
a split retainer ring comprising an outer tapered surface and configured to be positioned within the lower tapered chamber.
